(19) United States
(12) Patent Application Publication
(10) Pub. No.: US 2008/0312502 A1
(43) Pub. Date: Dec. 18, 2008

(54) System and Device for in Vivo Procedures

(76) Inventors: Christopher Paul Swain, London (GB); Elisha Rabinovitz, Haifa (IL)

Correspondence Address: Pearl Cohen Zedek Latzer, LLP, 1500 Broadway, 12th Floor, New York, NY, 10036, US

(21) Appl. No.: 12/095,129

(22) PCT Filed: Dec. 3, 2006
(86) PCT No.: PCT/IL06/01392

§ 371 Date: Aug. 5, 2008

Related U.S. Application Data
(60) Provisional application No. 60/741,465, filed on Dec. 2, 2005.
(60) Provisional application No. 60/801,387, filed on May 19, 2006.

(51) Int. Cl. A61B 1/06

(52) U.S. Cl. 600/118; 600/160

(57) ABSTRACT

The invention relates to a device system and method for providing images of an in vivo site during in vivo procedures, such as laparoscopy wherein the device is capable of illuminating an internal body cavity and has an immobilization unit to fasten the device to a desired location in vivo.

### FIELD OF THE INVENTION

The present invention relates to in-vivo imaging. More specifically the invention relates to a system, device and method for monitoring one or more in vivo sites during in vivo procedures.

### BACKGROUND OF THE INVENTION

In vivo sensing devices, such as thermometers, pH meters, optical scanners, image sensors and so on, can be used for unobtrusively monitoring body systems. During surgery or in the time immediately after surgery patients may experience organ functional problems. For example, during surgery in the gastrointestinal tract the blood pressure at the vicinity of the surgical site is reduced and peristalsis is arrested. After surgery the blood pressure increases and peristalsis is resumed sometimes causing bleeding from the surgical site into the intestine lumen.

Also, for example, in treating coronary artery disease, it is sometimes necessary to bypass coronary arteries with a vascular graft, which is surgically attached to the heart, to circumvent a blocked coronary artery. After surgery cardiac functional problems may occur due to build-up of stenotic lesions or other obstructions to the flow of blood through the implanted graft.

Postoperative monitoring of the digestive system is important to avoid letting too much time elapse before blood loss into the intestine is detected.

Similarly, it is important that the condition of a vascular graft be monitored, post-surgery, to detect the further build-up of stenotic lesions or other obstructions to the flow of blood through the implanted graft.

Various catheterization procedures are known for assessing the flow characteristics of a blood vessel or blood vessel graft. However, the introduction of catheters into the vascular system may result in damage to blood vessels.

Ultrasound echo imaging is known for visualization and examination of a patient's heart. However, methods of echocardiography do not always result in good quality images after cardiac surgery.

Monitoring of in vivo processes, not necessarily related to post surgical events, may also be an important diagnostic tool. For example, in endometriosis, in which cells that normally grow inside the uterus instead grow outside the uterus. Endometrial cells line the uterus and are normally shed each month during menstruation. When endometrial cells grow outside the uterus, the cells implant. These implants occur most commonly within the fallopian tubes and on the outside of the tubes and ovaries, the outer surface of the uterus and intestines and anywhere on the surface of the pelvic cavity. They can also be found, less often, on the surface of the liver, in old surgery scars or, very rarely, in the lung or brain. The implants cause internal bleeding, which leads to tissue inflammation and later, scarring and possibly infertility. Endometriosis can be suspected based on symptoms of pelvic pain and findings during physical examinations in the doctor's office but neither the symptoms nor the physical examination can be relied upon to establish the diagnosis of endometriosis. Imaging studies, such as ultrasound, can be helpful in studying the pelvis, but still cannot accurately diagnose endometriosis. Direct visual inspection and tissue biopsy of the implants are necessary for accurate diagnosis. Currently, the only accurate way of diagnosing endometriosis is at the time of surgery either by open standard laparotomy or laparoscopy.

Laparoscopy is a surgical procedure in which a specially designed scope and other surgical tools are inserted into the abdomen through a small incision. It is used for a variety of surgical procedures often for bladder, prostate, small intestine as well as fallopian tubes and pelvic cavity diagnosis and surgery. Laparoscopy requires direct visualization of the peritoneal cavity, ovaries, outside of the tubes and uterus. During a typical procedure, carbon dioxide (CO2) is put into the abdomen or other lumens through a special needle that is inserted from the out side. This gas helps in the initial separation the organs inside the abdominal cavity, this procedure is followed by an insertion of a trocar which is a hollowed tube with an inside diameter of 10-12 mm through which the surgeon can insert his tools. Typically the first procedure is CO2 pumping making it easier for the physician to see organs during laparoscopy. The gas is removed at the end of the procedure.

Typically three types of instruments called laparoscopes are used for visualization. The most common one is built like a telescope with a series of lenses and a light source. The other type is based on a bundle of optic fibers which bring light into the abdomen and carries the image outside. In some cases an image sensor is attached to the tip of a laparoscope that is inserted through the trocar into the body lumen created by the CO2 such that images of the body lumen can be displayed. The tip of sensor based laparoscope may be bendable to enable a larger field of view.

Laparoscopy may include several incisions in the abdomen in each of them a trocar is installed through which typically a variety of surgical or therapeutic tools are inserted (such as knifes, graspers, staplers etc.) but only one incision through which to visualize the surgical site. Although a bendable scope may enable a wide field of view, it still suffers from limited angles of view and limited camera maneuvering capabilities; it does not enable viewing behind a fold or on both sides of an organ. This may be important for example in a procedure of insertion of a needle from one side and taking it out from the other side during stitching. Additionally, the use of one imager suffers, inter alia, from the fact that in order to see details the camera must zoom in on a site, in which case the orientation for the surgeon is lost. Keeping orientation may be at the expense of being able to zoom in on details.

Flexible endoscopic based endoluminal and transgastric surgery for cholecystectomy, appendectomy, gastric reduction for obesity, and antireflux procedures show great promise for less invasive surgery. Current endoscopes and instruments are inadequate to perform complex surgeries. The lack of support for a retroflexed endoscope in the peritoneal cavity makes it hard to reach remote structures and makes the necessary vigorous retraction of tissues and organs such as the liver, stomach, gallbladder or intestines difficult. When flexible instruments are pushed against such structures the unsupported endoscope is pushed away without improving exposure.

### SUMMARY OF THE INVENTION

There is provided, in accordance with some embodiments of the present invention an in-vivo imaging system, device and method which may allow new angles of view in laparoscopy, improving efficacy and safety of the procedure.

In embodiments of the present invention, data relating to environmental conditions at an in vivo site, or images of an in vivo site, such as a surgical site, may be obtained over a specific period and thus embodiments of the invention enable monitoring of an in vivo site. For example, embodiments of the invention may enable post surgery monitoring of surgical operations, which provides camera or video images of the surgery site during the critical post surgery hours. The system, device and method of the invention may enable an external operator to directly observe changes occurring at an in vivo site, for example to monitor suspected endometriosis, such that pathological occurrences can be detected at their onset and immediately be treated.

Embodiments of the invention further provide a remote controlled immobilized or moveable in vivo sensing device that can be used for monitoring an in vivo site by utilizing any appropriate sensing device (for example, pH meter, blood detector, imaging device etc.). In the digestive system the device according to an embodiment of the invention may be immobilized or moved for monitoring a site in the digestive system without having to leave an opening in the patient or incise the patient twice for retrieval of the in vivo device at the end of the monitoring period.

In one embodiment the method comprises the steps of transiently immobilizing or moving an imaging device in the vicinity of a site in the digestive system; imaging the site and transmitting images of the site to an external workstation.

There is provided, in accordance with some embodiments of the present invention an in-vivo imaging system, device and method for monitoring one or more in vivo sites, for example imaging of biliary manipulations, and liver biopsy without the use of a conventional larascopy and imaging of subhepatic structures inaccessible to laparoscopy.

There is provided, in accordance with some embodiments of the present invention an in-vivo imaging system, device and method for placing an in-vivo sensing device, for example an in-vivo imaging device in the peritoneal cavity at laparoscopy to allow continuous wireless views of in vivo sites and target structures such as the gallbladder, diaphragm and the liver.

In some embodiments of the present invention, transabdominal and peritoneal attachment methods may be used for immobilization, and/or stabilization and remote manipulation of the in-vivo device.

A device according to one embodiment of the invention may be an autonomous, possibly single use imaging device. The imaging device, according to one embodiment may include an illumination source such as one or more LEDs for illuminating a site in vivo, an image sensor, such as a CCD or CMOS, for imaging the site in vivo and an optical system, which may include a lens or set of lenses, for focusing the image of the in vivo site on the image sensor. Typically, the image sensor, optical system and illumination source are enclosed within a housing of the imaging device. According to one embodiment the imager, optical system and illumination source are all positioned behind a viewing window, which may be part of the device housing and through which the in vivo site may be illuminated and imaged. Also included within the device housing may be a wireless transmitter for transmitting image data captured by the imager and/or other data obtained by the device and a power source, such as a battery, to power the components of the device.

According to some embodiments an imaging device may include two or more imagers and viewing windows, typically facing opposing directions for imaging a wider field of view.

Image (and other) data transmitted from the imaging device may be received outside a patient's body by a receiver placed on or near the patient's body. The data may then be transferred to a work station and may be displayed on a display of the workstation for a surgeon or other professional to view during or after a procedure. In some embodiment the receiver and workstation are integrated into one unit.

An imaging device according to embodiments of the invention may be tied to a thread or wire and may be introduced into a body lumen through an instrument such as an overtube, an endoscope or a trocar and may be manipulated to a desired site, such as a surgery site and may be fastened to that site by a grasper or any other appropriate tools that are inserted to the lumen either through another trocar or the same one.

According to one embodiment of the invention an in vivo imaging device may include tissue analysis capabilities. According to one embodiment the device may include an imager, such as a CMOS imager having a plurality of pixel arrays, typically one on top of the other. An imager according to this embodiment may enable the use of different wavelength to detect pathologies.

According to one embodiment several imaging devices may be used simultaneously in a single procedure and a combined image or separate images transmitted from each of the devices may be displayed simultaneously on the work station display, thus enabling multiple view points and as many viewing angles as necessary, all typically displayed on a single screen or on array of screens in front of the surgeons. Additionally, real images of the lumen may be supplemented with different band images that can give indications on pathologies such as tumors, intraluminal bleeding etc.

### BRIEF DESCRIPTION OF THE DRAWINGS

Embodiments of the invention are illustrated by way of example and not limitation in the figures of the accompanying drawings, in which like reference numerals indicate corresponding, analogous or similar elements, and in which:

FIG. 1 is a schematic illustration of an in-vivo imaging system according to an embodiment of the present invention;

FIG. 2A, is a schematic illustration of in-vivo imaging devices with immobilizing units, in accordance with embodiments of the present invention;

FIG. 2B shows an in-vivo device in association with the digestive system, in accordance with embodiments of the present invention;

FIG. 2C is a schematic illustration of an imaging device operable according to some embodiments of the present invention;

FIGS. 3A and 3B are schematic illustrations of an imaging device operable according to some embodiments of the present invention;

FIGS. 4A-4C are schematic illustrations of an imaging device operable according to some embodiments of the present invention; and

FIG. 5 is a schematic flow-chart of a method of imaging a body lumen, according to one embodiment of the present invention.

It will be appreciated that for simplicity and clarity of illustration, elements shown in the figures have not necessarily been drawn to scale. For example, the dimensions of some of the elements may be exaggerated relative to other elements for clarity.

### DETAILED DESCRIPTION OF TIE INVENTION

In the following description, various aspects of the present invention will be described. For purposes of explanation, specific configurations and details are set forth in order to provide a thorough understanding of the present invention. However, it will also be apparent to one skilled in the art that the present invention may be practiced without the specific details presented herein. Furthermore, well known features may be omitted or simplified in order not to obscure the present invention.

Embodiments of the invention can be utilized for monitoring, for example simultaneously, one or more in vivo sites in diverse body systems, as will be exemplified below. In one embodiment there is provided a system for monitoring sites in the GI tract, in which the sensing device is an imaging system.

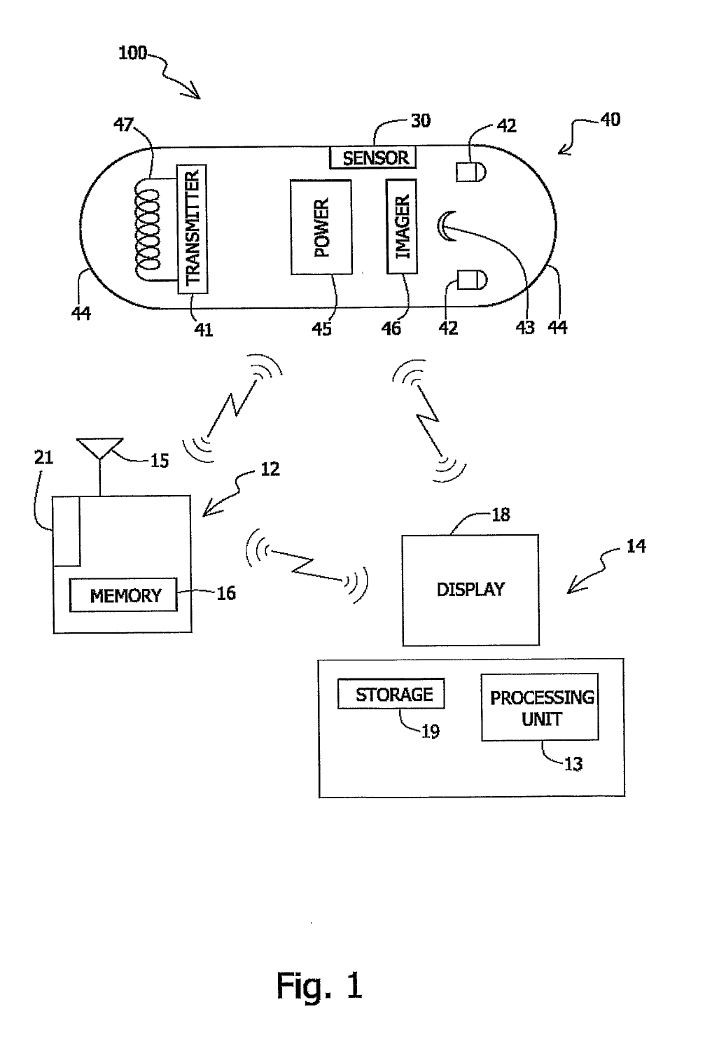

Reference is made to FIG. 1, which shows a schematic diagram of an in-vivo imaging system 100 according to one embodiment of the present invention. The in-vivo imaging system 100 may include an in-vivo imaging device 40 having, for example an imager 46, for capturing images, an optical system 43 for focusing images onto the imager, one or more illumination source(s) 42 such as a white LEDs (Light Emitting Diode), OLEDs (Organic LED) or other suitable illumination sources, for illuminating the body lumen. According to an embodiment of the invention the illumination source illuminates the body lumen through viewing window 44 and light rays from the body lumen are remitted to the imager through the viewing window 44. According to an embodiment of the invention the device also includes a power source 45 for powering device 40, and a transmitter/receiver 41 with antenna 47, for transmitting and/or receiving signals. Typically the transmitter transmits image data to an external device such as a receiver/recorder 12.

In some embodiments, imager 46 may include, for example, a CCD (Charge Coupled Device) camera or imager, a CMOS (Complementary Metal Oxide Semiconductor) camera or imager, a digital camera, a video camera, or other suitable imagers, cameras, or image acquisition components. In some embodiments, each frame of image data may include 256 rows, each row may include 256 pixels, and each pixel may include data for color and brightness according to known methods. According to other embodiments 320×320 pixel imager may be used. Pixel size may be between 3 to 10 micron. In another embodiment higher or lower resolution may be used. According to some embodiments pixels may be each fitted with a micro lens.

Transmitter/receiver 41 may operate using radio waves; but in some embodiments, transmitter/receiver 41 may transmit data via, for example, wire, optical fiber and/or other suitable methods. Other suitable methods or components for wired or wireless transmission may be used.

In one embodiment, all of the components may be sealed within the device body (the body or shell may include more than one piece); for example, the imager 46, the optical system 43, the illumination sources 42, the power source 45, the transmitter/receiver 41 and the antenna 47, may all be sealed within the device body.

In some embodiments of the present invention, in-vivo device 40 may include one or more sensors 30 other than and/or in addition to imager 46, for example, temperature sensors, pH sensors, pressure sensors, blood sensors, etc. In some embodiments of the present invention, device 40 may be an autonomous device. According to some embodiments the device is cylindrically shaped or may have a capsule shape.

Devices according to embodiments of the present invention, including imaging, receiving, processing, storage and/or display units suitable for use with embodiments of the present invention, may be similar to embodiments described in U.S. Pat. No. 5,604,531 to Iddan et al., entitled IN-VIVO VIDEO CAMERA SYSTEM, and/or in U.S. Pat. No. 7,009,634 to Iddan et al., entitled DEVICE FOR IN VIVO IMAGING and/or in U.S. patent application, Ser. No. 10/046,541 entitled A SYSTEM AND METHOD FOR WIDE FIELD IMAGING OF BODY LUMENS, all of which are assigned to the common assignee of the present invention and which are hereby incorporated by reference.

The in-vivo imaging device 40 may, according to some embodiments of the present invention, transmit information such as in-vivo image data or other data to the receiver/recorder 12 placed or installed within the range of the transmitting distance of device 40. The receiver/recorder 12 may include an antenna or antenna array 15 and a data storage unit or memory 16. The receiver/recorder 12 may of course take other suitable configurations and may not include an antenna or antenna array. In some embodiments of the present invention, the data receiver/recorder 12 may, for example, include processing power and/or a LCD display for displaying image data. In another embodiment receiver/recorder 12 is an integral part of the workstation 14.

According to some embodiments of the present invention, the receiver/recorder 12 may, for example, transfer the received data to a computing device 14, such as a workstation or personal computer, where the in-vivo image data may be further analyzed, stored, and/or displayed to a user. Typically, the image data is displayed substantially in real-time. According to some embodiments initial processing of the image data can be done in the imaging device itself or in the receiver to enable real-time viewing. According to other embodiments the data is stored in receiver/recorder 12 and is then downloaded to computing device 14 for off-line viewing by a professional. Computing device 14 may typically be a personal computer or workstation, which may include standard components such as a processing unit 13, a memory, for example storage 19, a disk drive, a monitor 18, and input-output devices, although alternate configurations are possible. Monitor 18 may be a conventional video display, but may, in addition, be any other device capable of providing an image, a stream of images and/or other data. Instructions or software for carrying out a method according to an embodiment of the invention may be included as part of computing device 14, for example stored in storage 19. In some embodiments, the receiver/recorder 12 may include a link 21 such as for example a USB, blue-tooth, radio frequency or infra-red link, that may connect to antenna 15 or to a device attached to antennas 15.

According to some embodiments of the present invention the memory 16 may be fixed in or removable from receiver/recorder 12. In some embodiments memory 16 may hold approximately 10 Gigabytes of memory.

According to one embodiment several imaging devices may be used concurrently. According to an embodiment of the invention transmission of data from the imaging device includes an identification signal which may be different for each individual device. Data received from the plurality of devices may thus be multiplexed, either at the receiver and/or in the workstation. Concurrent images may thus be displayed substantially concurrently on the computing device display 18. According to another embodiment image data transmitted from each device may include a time stamp to enable matching and displaying of concurrent images. According to one embodiment a single image may be displayed which is rendered from a plurality of concurrent image frames.

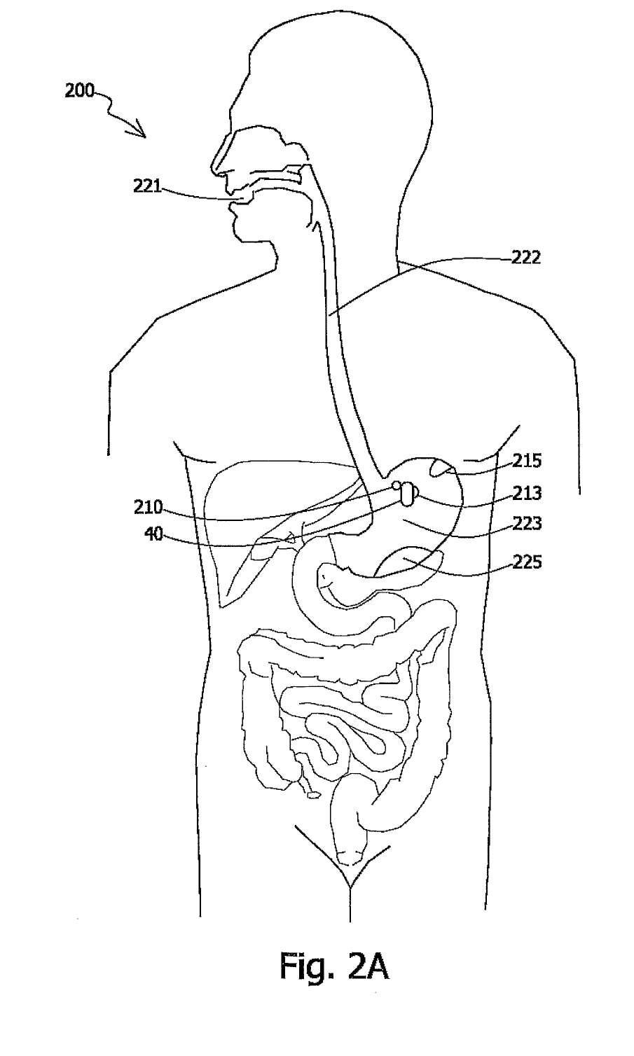

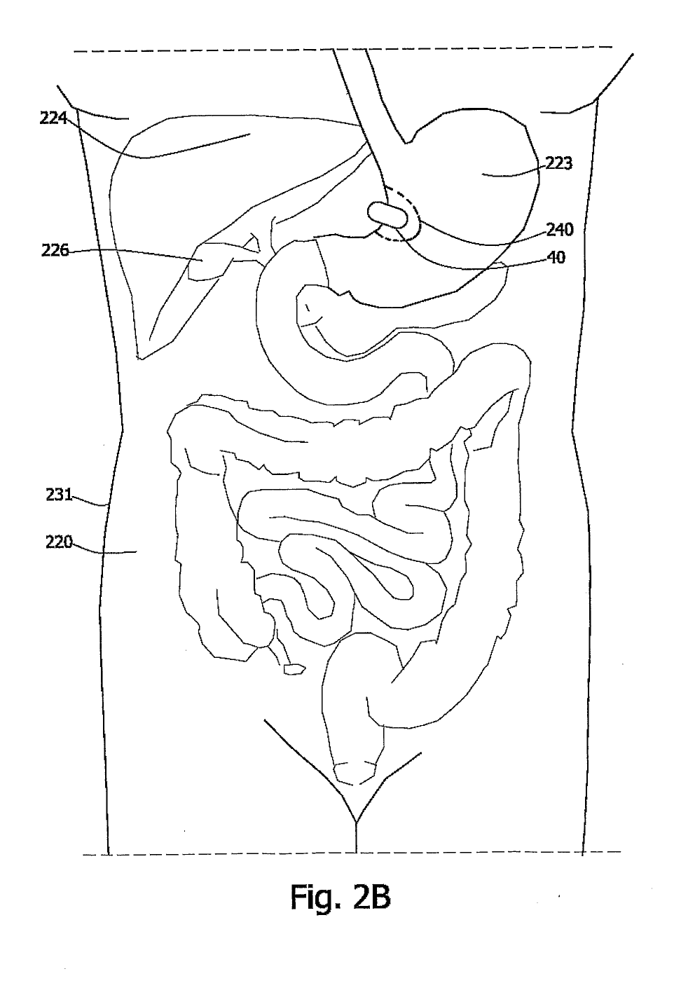

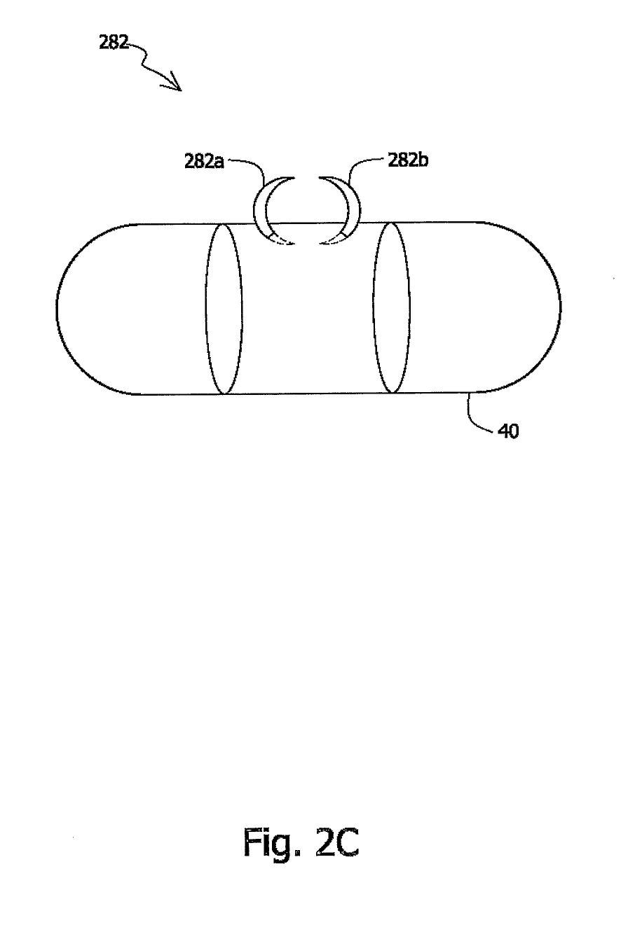

FIG. 2A shows an in-vivo device, such as the in-vivo imaging device 40 in association with human body 200 including mouth 221, esophagus 222 and stomach 223. Device 40 as depicted in FIG. 2A and according to one embodiment is generally capsule shaped, and may be easily swallowed and passively passed through the entire GI tract, pushed along, for example, by natural peristalsis. Nonetheless, it should be noted that device 40 may be of any shape and size suitable for being inserted into and passing through a body lumen or cavity, such as spherical, oval, cylindrical, etc. or other suitable shapes. Furthermore, device 40 or various embodiments that may include at least some components of device 40 may be attached, tied or affixed on to an instrument that is inserted into body lumens and cavities, such as, for example, on an endoscope, laparoscope, needle, catheter an overtube etc.

The imaging system 100 may provide direct visual information of an in vivo site (i.e., a surgical site) such that visibly detectable changes at the site, such as bleeding, swelling etc. can be seen by an external operator. The in-vivo device 40 may further be in communication with a processor/control for analyzing the data detected by it and possibly for controlling the sensing device. According to some embodiments of the present invention, images of a surgical site may be transmitted to a processor where they are analyzed for the presence and possibly the concentration of blood (for example, by detecting certain changes in color). The image may then be received by the receiver/recorder 12 and viewed by an external operator including additional information, generated by the processor, regarding the bleeding at the surgical site. Further, the system may include means for alerting the external operator. The means for alerting the external operator are in communication with the processor. Thus, when the presence of blood is detected by the processor a signal, such as a flashing light or an alarm, may be activated to alert the external operator.

For example, in the time immediately after surgery an in-vivo device such as device 40 may be introduced into to a body lumen, such as the stomach 223, to monitor one or more in-vivo sites e.g. sites 215 and 225. Site 215 may be a bleeding surgical site at the top of the stomach, while site 225 may be at the bottom of the stomach e.g. where the blood from the bleeding surgical site is accumulated.

According to some embodiments of the present invention the device 40 may include one or more rings such as ring 213, on its perimeter. The ring 213 may fit into depressions in the device body perimeter so that they do not protrude from the device 40 perimeter and do not obstruct the device's passage through the GI tract. The ring 213 may be used for sewing the device 40 to a desired location in vivo, for example in the vicinity of a surgical site in the GI tract. Following a surgical operation in the GI tract the surgeon may place the device 40 at a location in the vicinity of the surgical site with the viewing window (for example, viewing window 44) facing the surgical site. The surgeon then immobilizes the capsule to its place by sewing a suture through ring 213 and through the tissue at the location. The device 40 is thus fastened to the tissue from two sides of the device 40. It should be appreciated that one or more rings can be used in the invention, depending, inter alia, on the shape of the housing and the anatomy of the in vivo site.

The in-vivo device 40 may be preprogrammed to image the surgical site at any desired rate. Optionally, the rate of obtaining images can be controlled either by an external operator or by a processing/controlling unit, as described above, wherein detection of blood, for example, will cause an increased rate of image acquisition.

According to some embodiments the device 40 may include one or more imagers, for example two imagers which may capture images of one or more in-vivo sites such as sites 215 and 225, simultaneously, and may transmit images of the surgical sites e.g. sites 215 and 225 to a the receiver/recorder 12 in real time or in constant time intervals. The in-vivo device 40, according to embodiments of the invention, may be immobilized in the vicinity of an in vivo site. For example the device 40 may be attached to an attachment point 210 e.g. to the stomach tissue to monitor and/or to measure, for example during surgery in the gastrointestinal tract, the blood pressure at the vicinity of the surgical site.

FIG. 2B shows an in-vivo device, such as the in-vivo imaging device 40 in association with a patient's abdominal cavity 220 including the stomach 223, liver 224 and the gallbladder 226. The device 40 may be introduced to the body lumen (e.g., the abdominal cavity) for example through the abdominal wall 231 using a PEG (Percutaneous Endoscopic Gastrostomy) tube or through the mouth e.g. by an endoscope, and may be placed for example at an opening 240 in the stomach tissue. For example the device 40, which may include imagers at two opposing ends of the device, may be passed through a transgastric incision and may simultaneously view both sides of the gastric incision, for example during suturing maneuvers. In addition, placing the imaging device at the opening 240 may allow imaging of organs in the abdominal cavity such as the liver 224 and the gallbladder 226, thus enabling e.g. imaging of biliary manipulations, and liver biopsy without the use of a conventional larascopy image and imaging of subhepatic structures inaccessible to laparoscopy.

According to some embodiments an in-vivo device may be used as a monitoring tool during an in-vivo procedure, for example to illuminate and/or to image an in-vivo site during surgery. An in-vivo device such as device 40 may be inserted into the abdominal cavity through an instrument such as an endoscope, a PEG tube or a trocar. Before surgery or during surgery, carbon dioxide (CO2) is put into the abdomen or other lumens through a special needle that is inserted from the out side. This gas helps in the initial separation the organs inside the abdominal cavity. This procedure is followed by an insertion of a trocar which is a hollowed tube with an inside diameter of 10-12 mm through which the surgeon can insert his tools. Typically the first procedure is CO2 pumping making it easier for the physician to see organs during laparoscopy. The gas is removed at the end of the procedure.

As shown in FIG. 2C, some embodiments of the invention may include an immobilization unit 282 housed within or attached to an autonomous imaging device, such as device 40. An embodiment of the immobilization unit 282 may include a pair of grasping tongs or arms 282a and 282b. The grasping aims 282a and 282b may be made of a wide variety of biocompatible materials, for example, stainless steel or a rigid plastic, for example, a rigid polymer. Although the arms illustrated in this embodiment are crescent shaped, the arms or grasping tongs may take a wide variety of conformations for the purposes of the present invention. One embodiment of such an immobilizing unit may be used to fasten the device to a desired spot within the body lumen. According to other embodiments an autonomous imaging device may be attached to or fitted on a unit such as a grasper. The grasper may then be attached to a tool such as a knife or scissor thus providing close-up view of the cut as it is being formed. According to further embodiments, an imaging device may be attached to a trocar tube from its out side without blocking the trocar opening such that a tool inserted and operated through that trocar may be viewed by the operator. According to other embodiments an autonomous imaging device may be attached to or fitted on a unit such as a grasper. The grasper may then be attached to a tool such as a knife or scissor thus providing close-up view of the cut as it is being formed.

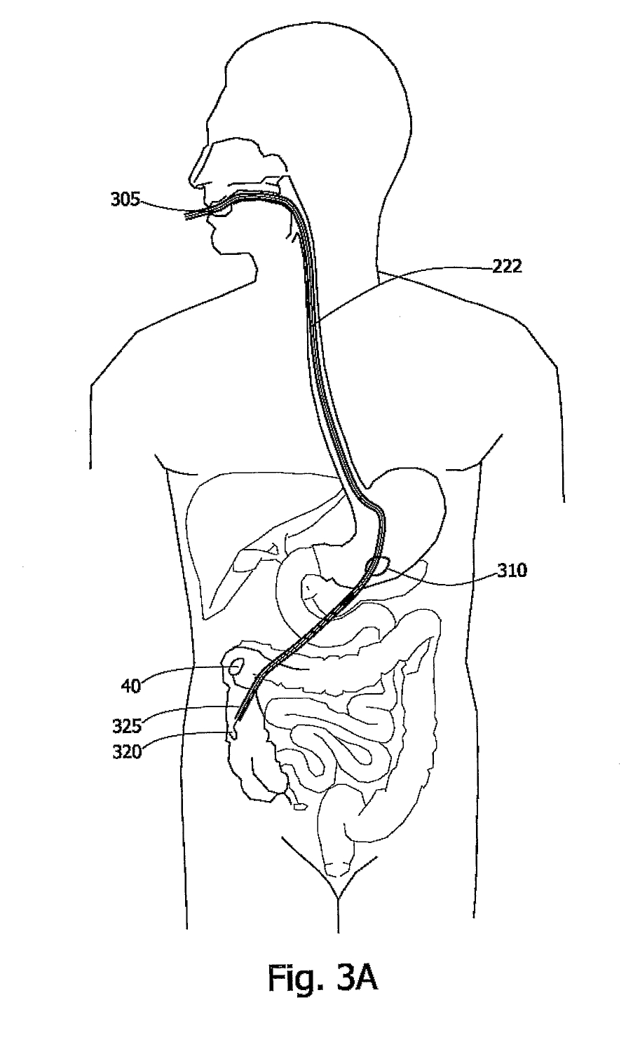

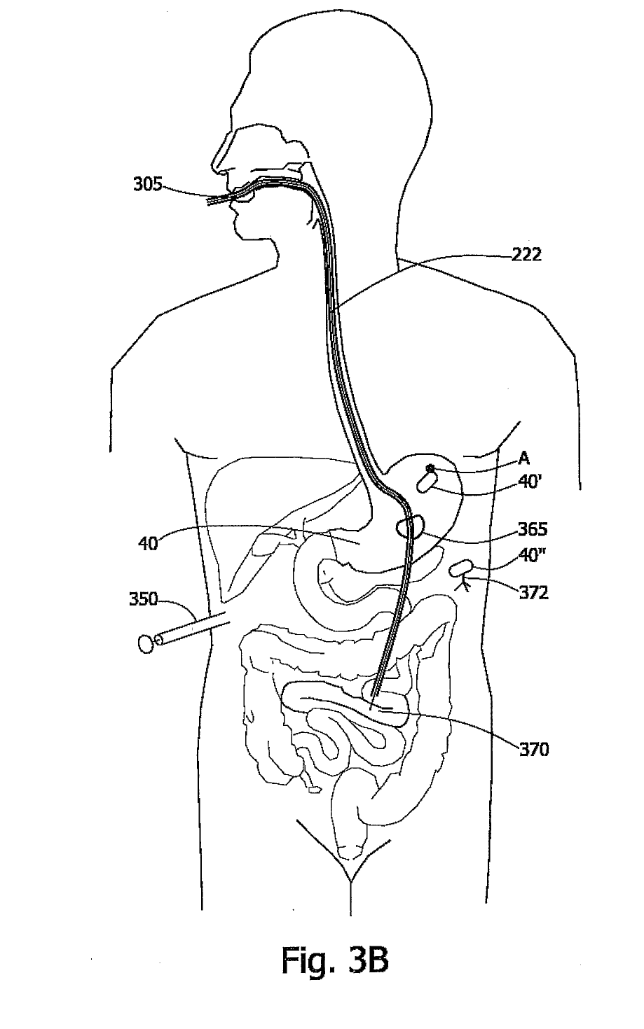

According to some embodiments of the present invention, as shown in FIG. 3A, an in-vivo device, such as device 40, may be inserted into the abdominal cavity, for example during an appendix removal surgery, to monitor a surgical site, e.g. site 325, and to aid a surgeon to easily navigate his endosurgical device tools e.g. in the overtube 305, to remove the body lumen part 320 causing the appendicitis. The device 40 may be attached to an edge of the overtube 305, leaving the total overtube 305 inner diameter available for endosurgical device passage. According to some embodiments, the device 40 may be introduced to a surgical site in the abdominal cavity for example through the stomach wall after needle-knife incision e.g. through an opening such as opening 310.

According to some embodiments of the present invention, as shown in FIG. 3B, one or more in vivo devices such as devices 40′ and 40″ may be used for monitoring in vivo sites during, for example, an Obesity Surgery which may involve reducing the size of the stomach or shortening the small intestine in order to change the way the body digests and absorbs food. Before the surgery or during the surgery, carbon dioxide (CO2) is put into the abdomen or other lumens through a special needle, e.g. needle 350 that is inserted from the out side of the body. A first in-vivo device, such as device 40′, may be introduced into a body lumen, through the mouth for example by an endoscope or an overtube e.g. overtube 305 and may be attached for example to spot A. The in-vivo device 40′ may be used for monitoring one of the overtube surgery tools while it cuts an opening 365 in the stomach. The first imaging device 40′ or a second in-vivo device 40″ may be inserted into the abdominal cavity by the overtube 305 or by other instruments and may monitor a second in vivo site. For example the second in-vivo device 40″ may be used to monitor the abdominal cavity and to aid the surgeon in pulling a portion of the small intestine e.g. portion 370, for example 25 cm of the small intestine, and to insert the portion 370 into the stomach through the opening 365. In some embodiments the device 40″ may be attached to a tripod, such as tripod 372 which may include an immobilizing unit (e.g., the three legs of the tripod) which may be used to fasten the device to a desired spot within the body lumen. In some embodiments the tripod 372 may include a mechanism to push body lumen parts for example the stomach wall or the abdominal wall making it easier for the physician to see organs during laparoscopy. The mechanism (not shown) may include an additional arm for pushing body parts.

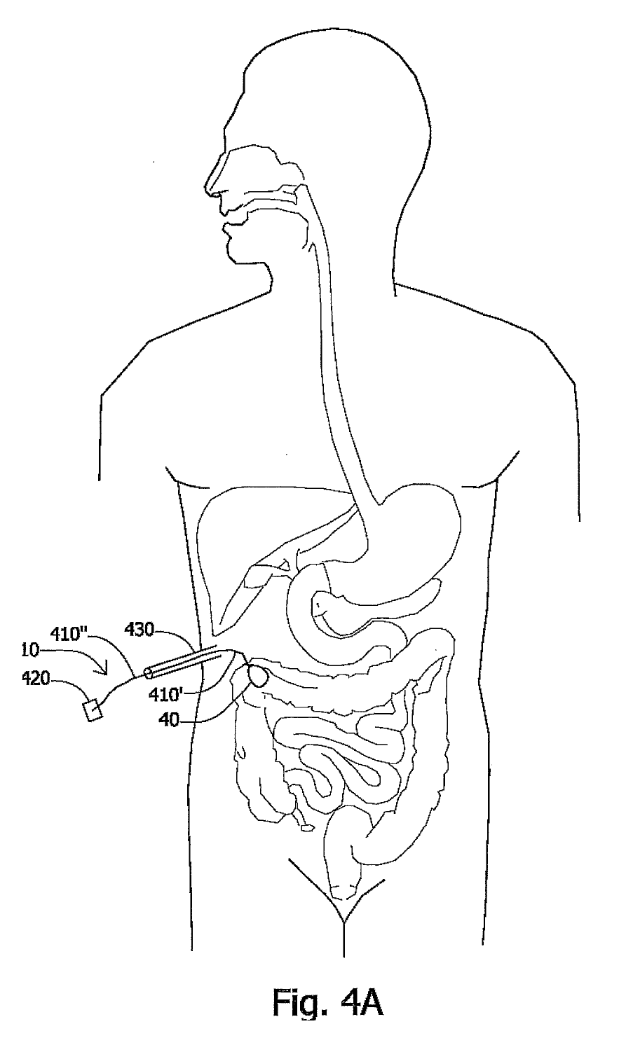

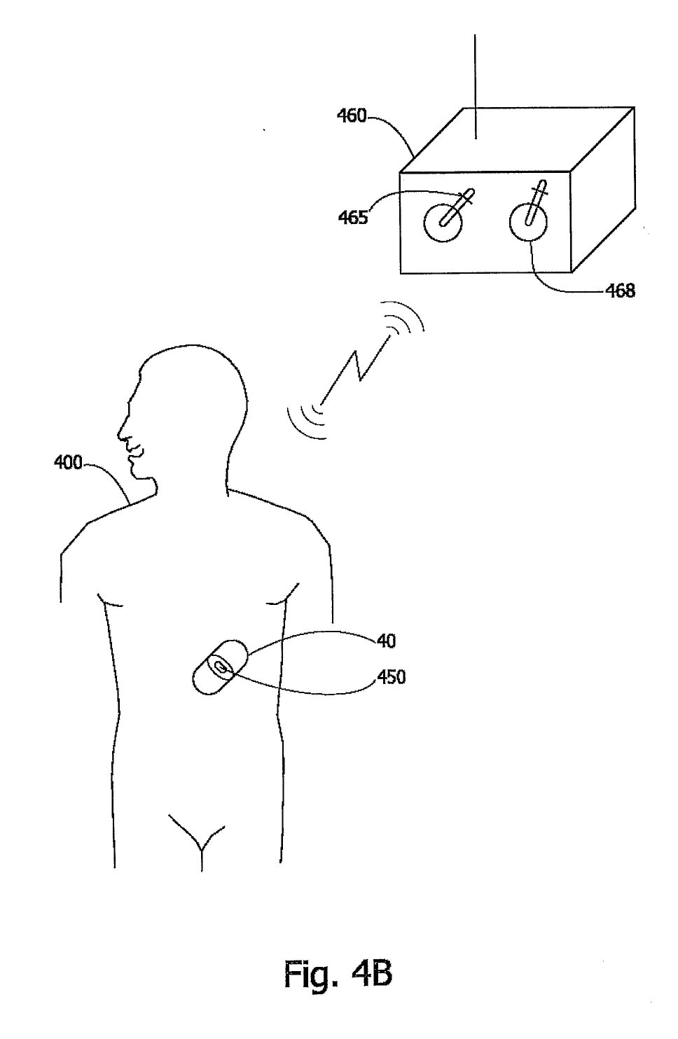

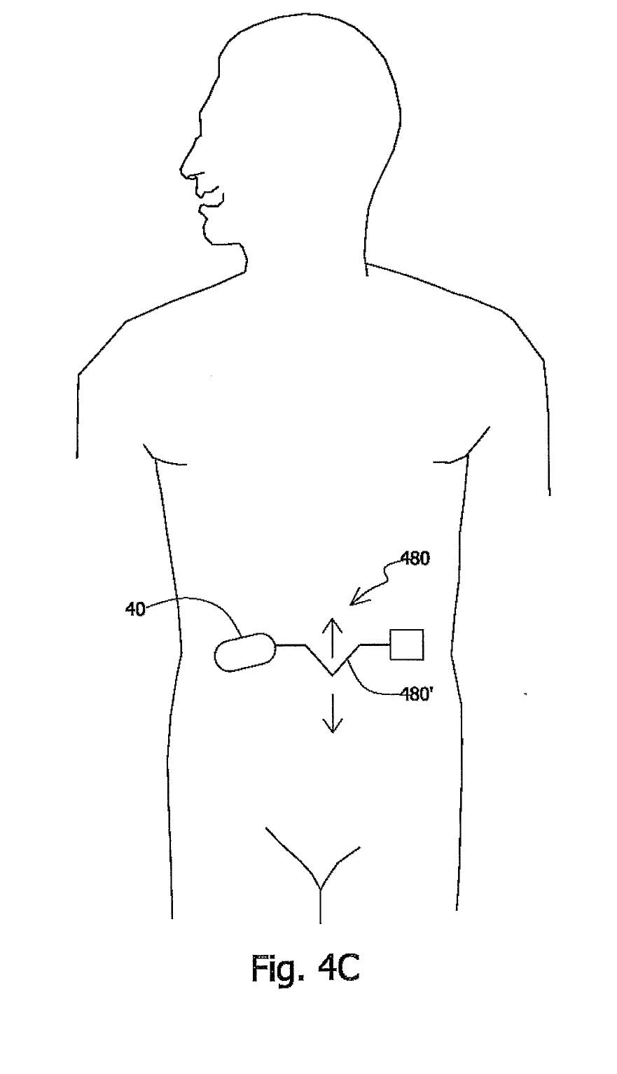

According to some embodiments of the present invention, an in-vivo device such as the devices 40 shown in FIGS. 1, 2A, 2B, 3A and 3B may be angled, rotated and/or may be moved, e.g. lowered or raised, in the body lumen e.g. the abdominal cavity or the stomach, to capture multiple images of specific targets. For example, as shown in FIG. 4A, the device 40 may be tied to a first end 410′ of a wire or thread such as wire 410, and may be inserted into the abdominal cavity through an instrument such as a PEG tube 430. A physician may manually control the movement of the device 40 inside the abdominal cavity by rotating pulling or pushing the second end 410″ of the thread outside the body.

According to some embodiments of the present invention, PEG tube 430 may be used to inflate, for example the stomach or bowel. The device 40 may be attached, e.g. tied, to wire 410 and may be inserted into the body, for example through the mouth. The device 40 may be raised and/or lowered, for example up and down the small intestine. This would allow the device 40 to be stopped at a specific location for surgery (e.g. biopsy, coagulation or even bile duct and pancreatic duct cannulation).

In some embodiments a motor such as motor 420, may be connected outside the body to the edge 410″ and may enable the movement of the device 40 inside the body, for example to change the direction of view of the device 40. The device 40 may be moved in the body lumen by a surgeon during a surgery and may improve views of less accessible targets in the body lumen, which could not be obtained with conventional endoscopes. In some embodiments a wire, such as wire 410 may be used for transmitting/receiving images and/or energy to the in-vivo imaging device 40.

According to another embodiment of the present invention, as shown in FIG. 4B, the device 40 may be wirelessly controlled from outside the body 400. For example, the device 40 may include a motor 450 and/or a servo which may be wirelessly controlled with RF, sound, IR (Infra-Red) or ultrasound by a remote control device 460. The remote control may include, for example, a first joy stick 465 for moving the device up-down and left-right and a second joy stick 468 for rotating the device 40 in the body.

According to another embodiment of the present invention, as shown in FIG. 4C, the device 40 may be attached to an apparatus 480 which may include a shape memory material. Shape memory materials known in the art are capable of a conformational change in response to sound, temperature changes e.g. usually in response to a temperature above and/or below the material's transition temperature for example in response to heat which may be created by passing an electric current through conducting wires. The shape memory material, which can be any of the suitable known shape memory alloys or shape memory polymers, is incorporated, according to an embodiment of the invention, into the apparatus 480 so as to enable deflection of the device 40, for example in the abdominal cavity. For example, in various embodiments, substantially the entire apparatus 480 or any portion of the apparatus, such as the portion 480′, or strips along the apparatus, are made of a shape memory material. The shape memory materials can be bent to various configurations in response to changes in temperature or in response to sound. Thus, different natural or induced in vivo environments having different temperatures can be used to deflect an in vivo sensor in various directions thereby achieving flexibility and, according to one embodiment, enabling the device 40 a wide angle of view of the body lumen, for example during a procedure e.g. an Obesity Surgery.

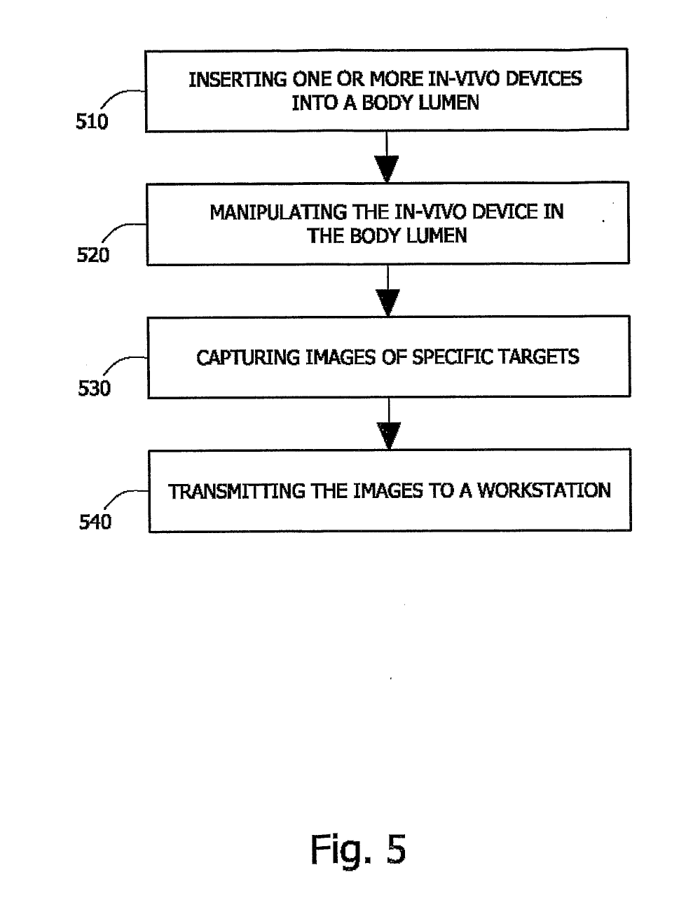

FIG. 5 is a schematic flow-chart of a method for obtaining data at an in vivo site, e.g. a surgical site, over a specific period of time and thus enabling monitoring an in vivo site, according to some embodiments of the present invention. In operation 510, one or more in vivo devices such as the device 40 may be inserted into a body lumen. For example the in-vivo device 40 may be passed through transgastric incisions, or through and beyond esophageal overtubes leaving the total overtube inner diameter available for endosurgical device passage. In operation 520 the in-vivo device may be manipulated in a body lumen. For example, the in-vivo device may be attached to a body lumen wall and may be manipulated to change orientation while it is attached to the wall. In some embodiments the in-vivo device may be manipulated, e.g. moved raised, lowered, rotated or angled, in a body lumen for monitoring one or more specific in vivo sites e.g. a bleeding area or a surgical site. For example, according to one embodiment the in-vivo device 40 may be sewn to the stomach wall or held by a thread. In another embodiment the device 40 may include a motor or servo and may be wirelessly controlled to move in the body lumen. In operation 530 the in-vivo device 40 may capture images of specific targets. For example, the in-vivo device 40 may capture simultaneous images of both sides of a gastric incision during suturing maneuvers or of the lower esophageal junction, ulcers, and biliary orifice. In operation 540 the images may be transmitted to a workstation where they may be for example analyzed and viewed.

It will be appreciated by persons skilled in the art that the present invention is not limited to what has been particularly shown and described hereinabove. Alternate embodiments are contemplated which fall within the scope of the invention.

1-24. (canceled)

25. A system for providing direct visual information of at least one side of in vivo incision sides of an in vivo incision, comprising:
an autonomous in vivo device comprising:
an immobilization unit to fasten the device through an in vivo incision;
at least one imager for imaging at least one side of the incision;
at least one illumination source for illuminating the at least one side of the incision; and
a transmitter for transmitting images of the side of in vivo incision; and
a receiver for receiving the transmitted images.

26. The system according to claim 25, wherein said immobilization unit comprises grasping tongs.

27. The system according to claim 25, wherein said transmitter is a wireless transmitter.

28. The system according to claim 25, further comprising a processing unit for controlling the image acquisition rate of the in-vivo device.

29. The system according to claim 25, wherein said imager is configured for further imaging an in-vivo site selected from the group consisting of: the lower esophageal junction, ulcers, biliary orifice, and liver.

30. The system according to claim 25, wherein said in vivo device is configured to monitor blood pressure at the vicinity of the in-vivo site.

31. A method for monitoring at least one in-vivo site over a specific period of time, the method comprising:
performing an incision in a body lumen wall;
introducing at least one in vivo device through the incision, said device comprising an immobilization unit and an imager for imaging at least one side of the incision;
attaching the in-vivo device to the body lumen wall at one side of the incision; and
manipulating the in-vivo device to change its orientation while said device is attached to the body lumen wall.

32. The method according to claim 31, comprising capturing an image of an in-vivo site selected from the group consisting of: the lower esophageal junction, ulcers, biliary orifice, and liver.

33. The method according to claim 31, comprising sewing the in-vivo device to a stomach wall.

34. The method according to claim 31, comprising wirelessly transmitting images of the in-vivo incision to a receiver in real time.

35. The method according to claim 31, comprising controlling the image acquisition rate.

36. The method according to claim 31, comprising introducing the in-vivo device to the abdominal cavity using a Percutaneous Endoscopic Gastrostomy tube.
